(19) United States
(12) Patent Application Publication
(10) Pub. No.: US 2017/0304091 A1
(43) Pub. Date: Oct. 26, 2017

(54) BIODEGRADABLE STENT

(71) Applicant: TERUMO KABUSHIKI KAISHA, Tokyo (JP)

(72) Inventors: Takashi KUMAZAWA, Fujinomiya-city (JP); Haruhiko KAMIJO, Hadano-city (JP); Hiroaki NAGURA, Hadano-city (JP)

(73) Assignee: TERUMO KABUSHIKI KAISHA, Tokyo (JP)

(21) Appl. No.: 15/646,625

(22) Filed: Jul. 11, 2017

Related U.S. Application Data
(63) Continuation of application No. PCT/JP2016/050373, filed on Jan. 7, 2016.

(30) Foreign Application Priority Data
Jan. 13, 2015 (JP) 2015-004312

(51) Int. Cl. A61F 2/88; A61L 31/16; A61L 31/08; A61L 31/14; A61L 31/06; A61K 31/436

(52) U.S. Cl. CPC A61F 2/88; A61L 31/06; A61K 31/436; A61L 31/08; A61L 31/148; A61L 31/16; A61F 2310/0097; A61F 2230/0069; A61F 2210/0004; A61L 2300/416

(57) ABSTRACT

A biodegradable stent that is degraded in a living body includes a stent body that is made of a biodegradable material and is deformed to have an expanded diameter in the living body; and a biodegradable drug-coating portion formed on the stent body. The drug-coating portion is degraded, in an expansion retention period during which an expansion retention force (radial force) of the deformed diameter-expanded stent body that acts on an inner wall of a lumen thereof is maintained, and before 60% of a degradation period from indwelling of the stent body in the living body to complete degradation thereof elapses.

### CROSS-REFERENCES TO RELATED APPLICATIONS

This application is a continuation of International Application No. PCT JP2016/050373 filed on Jan. 7, 2016, and claims priority to Japanese Patent Application No. 2015-004312, filed on Jan. 13, 2015, the entire content of each of which is incorporated herein by reference.

### TECHNICAL FIELD

The present invention relates to a biodegradable stent as a medical device.

### BACKGROUND

A stent is a medical device used to expand a stenosed site or obstruction site so as to secure a lumen, in order to treat various disorders due to a stenosis or obstruction in a lumen of a blood vessel. As such a stent, a biodegradable stent provided with a drug-coating portion has been known (refer to PTL 1 below). One kind of stent used in such procedures is a biodegradable stent provided with a drug-coating portion, such as disclosed in JP-A-H06-218063.

A stent formed of non-biodegradable metal will not naturally degrade after being indwelled in a living body. Instead, the stent continues indwelling in the living body until such time as a removal procedure is performed. Therefore, in treatments which use such a stent, there is a concern about safety, patient discomfort, or the like from long-term indwelling. By comparison, since a biodegradable stent is configured to be naturally degraded in the living body after a predetermined indwelling period elapses, the biodegradable stent is more beneficial than the non-biodegradable stent in terms of safety or patient discomfort from the long-term indwelling.

With a biodegradable stent provided with the drug-coating portion, in order to maintain a state in which the lumen as a treatment target is expanded for a predetermined period from a start of the indwelling, it is preferable that an expansion retention force (radial force) maintain a certain level, and it is preferable that the drug-coating portion is degraded and disappears as early as possible after drug is eluted (released) so as to exhibit desired drug efficacy, regarding inhibition of evocation of inflammatory response or delay in a healing process of luminal endothelium.

### SUMMARY

However, with a biodegradable stent in the related art such as disclosed in JP-A-H06-218063, a relationship between a degradation period (period of time from a start of indwelling in the living body to degradation (disappearance) in the living body) of the drug-coating portion and a period during which the stent maintains the radial force (expansion retention force), and a relationship between those periods and a degradation period of a stent body that configures a stent body portion are not particularly defined. Therefore, there still remains room for improvement in terms of improvement of treatment effects.

An object of the present disclosure is to provide a biodegradable stent that has a defined relationship between a degradation period of a drug-coating portion, a period during which a stent body that configures a stent body portion maintains an expansion retention force, and a degradation period of the stent body, and thus exhibits high treatment effects.

A biodegradable stent according to the present disclosure that achieves the object described above is degraded in a living body, the biodegradable stent including: a stent body that is made of a biodegradable material and is deformed to have an expanded diameter in the living body; and a biodegradable drug-coating portion formed on the stent body. The drug-coating portion is degraded, in an expansion retention period during which an expansion retention force of the deformed diameter-expanded stent body that acts on an inner wall of a lumen in the living body is maintained, and before 60% of a degradation period from indwelling of the stent body in the living body to complete degradation thereof elapses.

The biodegradable stent according to the present disclosure includes the drug-coating portion that exhibits desired drug efficacy and is degraded in the expansion retention period during which the stent body maintains the expansion retention force (radial force). In addition, after the drug-coating portion exhibits the desired drug efficacy, the drug-coating portion is rapidly degraded before the stent body is degraded, whereas the stent body maintains the expansion retention force over a predetermined period even after the drug-coating portion is degraded. Hence, according to the present disclosure, it is possible to provide the biodegradable stent that can suitably maintain, over the desired period, a state in which the lumen is widened, and improves treatment effect due to suitable exhibition of drug efficacy.

### BRIEF DESCRIPTION OF THE DRAWINGS

FIG. 1 is a view of a stent according to an embodiment. FIG. 1(A) is a perspective view illustrating an overview of the stent, and FIG. 1(B) is a development view of the stent.

FIG. 2 is an enlarged cross-sectional view of a part of a strut of a stent according to the embodiment. FIG. 2(A) is a cross-sectional view illustrating an example of a configuration of a drug-coating portion formed only on an outer surface of the strut, and FIG. 2(B) is a cross-sectional view illustrating another example of a configuration of the drug-coating portion formed on the outer surface and a side surface of the strut.

FIG. 3 is a diagram illustrating a relationship between an indwelling period of the stent, a residual amount of each portion, and a change in an expansion retention force according to the embodiment.

FIG. 4 is a view for illustrating action of the stent according to the embodiment, as a cross-sectional view schematically illustrating a state in which the stent is inserted in a lumen (blood vessel).

FIG. 5(A) is a cross-sectional view schematically illustrating the action of the stent in a state of indwelling in the lumen at a time between time T1 and time T2 in FIG. 3, and FIG. 5(B) is an enlarged cross-sectional view illustrating portion 5B in FIG. 5(A).

FIG. 6(A) is a cross-sectional view schematically illustrating the action of the stent in a state of indwelling in the lumen at time T2 in FIG. 3, and FIG. 6(B) is an enlarged cross-sectional view illustrating portion 6B in FIG. 6(A).

FIG. 7(A) is a cross-sectional view schematically illustrating the action of the stent in a state of indwelling in the lumen at time T3 in FIG. 3, and FIG. 7(B) is an enlarged cross-sectional view illustrating portion 7B in FIG. 7(A).

FIG. 8(A) is a cross-sectional view schematically illustrating the action of the stent in a state of indwelling in the lumen at time T4 in FIG. 3, and FIG. 8(B) is an enlarged cross-sectional view illustrating portion 8B in FIG. 8(A).

FIG. 9(A) is a cross-sectional view schematically illustrating the action of the stent in a state of indwelling in the lumen at time T5 in FIG. 3, and FIG. 9(B) is an enlarged cross-sectional view illustrating portion 9B in FIG. 9(A).

### DETAILED DESCRIPTION

Hereinafter, an embodiment of the present disclosure will be described with reference to the accompanying figures. Note that the following description does not limit a scope or meanings of terms described in Claims. In addition, dimension ratios in the figures increase, depending on the description, and thus the ratios are different from actual ratios in some cases.

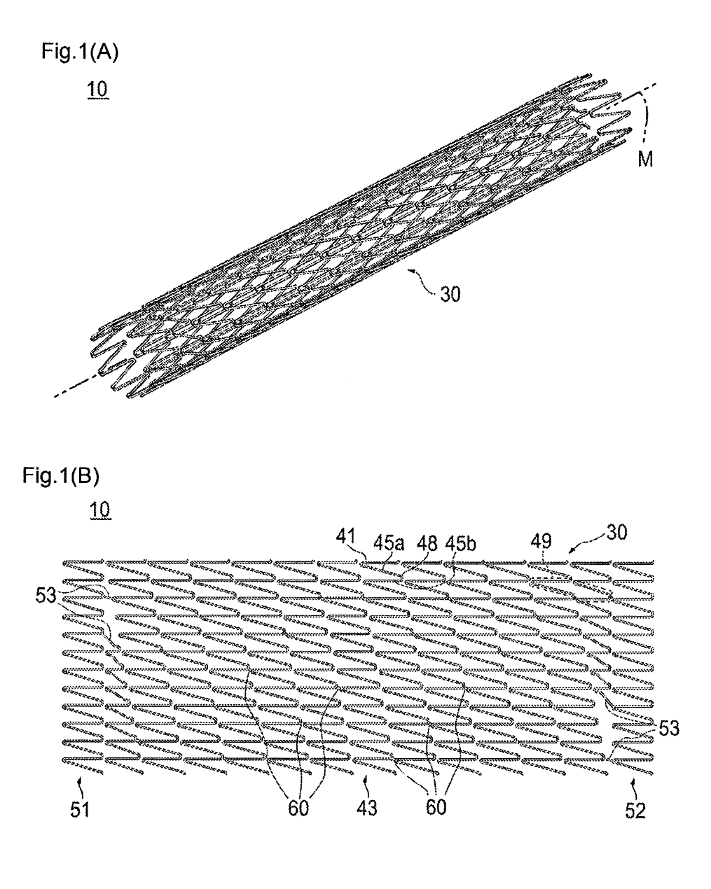

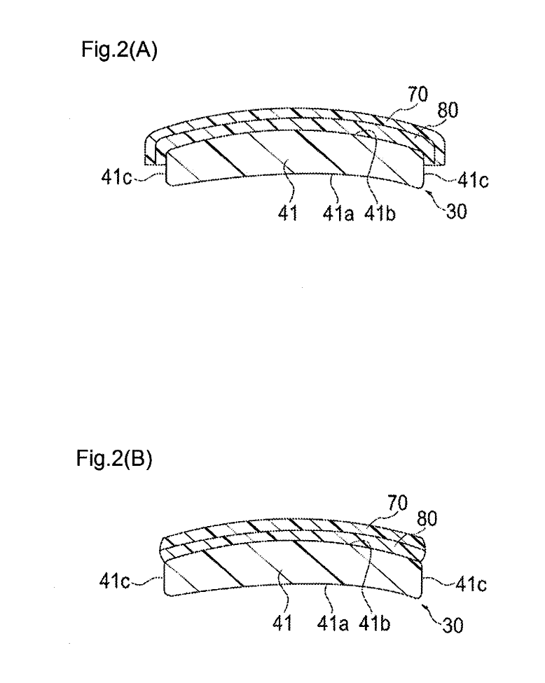

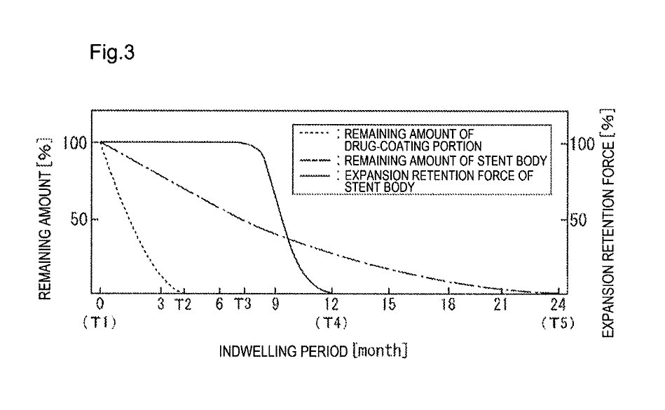

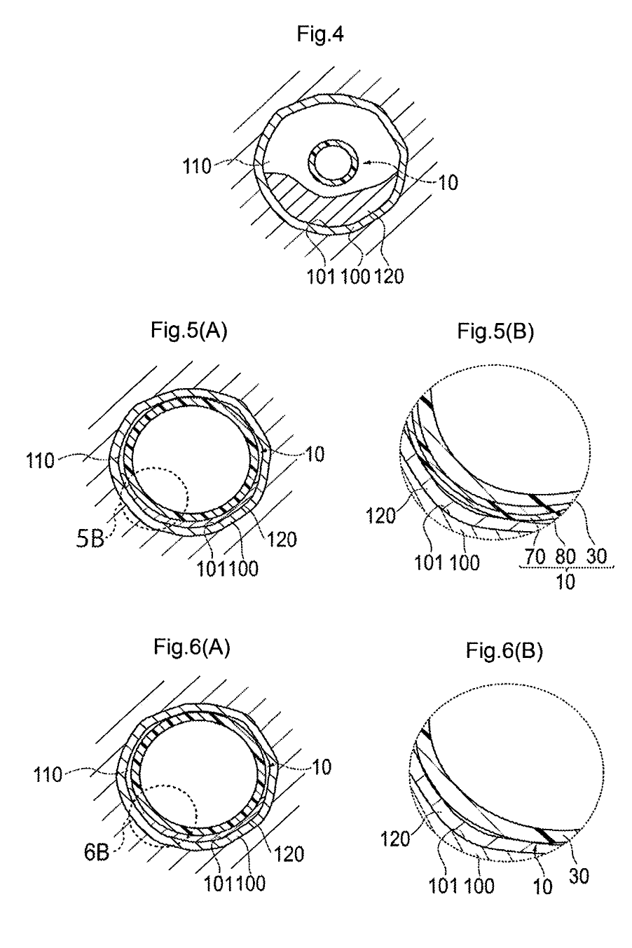

FIGS. 1 and 2 are views provided for illustrating a configuration of a stent according to an embodiment, FIG. 3 is a diagram illustrating a relationship between an indwelling period of the stent, a residual amount of each portion, and a change in an expansion retention force according to the embodiment, and FIGS. 4 to 9 are views provided for illustrating action of the stent according to the embodiment. Note that, in the description of the specification, a longitudinal direction (horizontal direction in FIG. 1(B)) of the stent is referred to as an axial direction represented by an axial line M.

First, a configuration of portions of a stent 10 is described. Note that, a configuration of the stent 10 illustrated in the figure is an example, and the stent of the present invention is not limited to a shape or a structure (for example, arrangement or design of struts) described here.

As illustrated in FIGS. 1(A) and 1(B), the stent 10 according to the embodiment includes a stent body (stent body portion) 30 formed of coil-shaped struts (linear configurational element) 41 that are integrally continuous, and has a substantially cylindrical external shape formed to have a predetermined length in an axial direction as a whole. The stent 10 indwells in a lumen (for example, a blood vessel, a bile duct, a trachea, an esophagus, another gastrointestinal tract, or a urethra) of a living body and widens the lumen, thereby being used in order to achieve medical treatment of a stenosed site or a obstructed site. In addition, the stent 10 indwells by being deformed to have an expanded diameter by a balloon provided in a balloon catheter, and is configured as a so-called balloon expandable stent. However, the stent 10 may be configured as a self-expandable stent that self-expands such that the stent body 30 has a predetermined diameter-expanded shape stored in advance after a start of indwelling. Furthermore, the stent may be a scaffold, such as a bioresorbable coronary scaffold, that is absorbed into the living body as it degrades.

The stent 10 is a biodegradable stent that is degraded and absorbed in a living body. The stent body 30 provided in the stent 10 is made of a biodegradable material and indwells in the living body in a state of being deformed to have an expanded diameter (refer to FIG. 5).

As illustrated in FIG. 1(B), a strut 41 is turned back to have a wave shape in the axial direction (longitudinal direction) of the stent body 30 and is provided with a plurality of spiral portions 43 extending to form a spiral shape around the axial direction (circumferential direction) of the stent body 30, and endless annular portions 51 and 52 disposed at both end portions in the axial direction of the stent body 30.

The spiral portion 43 and the annular portions 51 and 52 are integrally formed in the stent body 30 so as to configure a part of the stent body 30. The adjacent spiral portions 43 are connected to each other via a predetermined connection section 60 made of a polymer material or the like. In addition, the annular portions 51 and 52 are connected via a link portion 53 to the spiral portions 43 adjacent to the annular portions. The link portion 53 is integrally formed in the stent body 30 along with the spiral portion 43 and the annular portions 51 and 52.

As illustrated in FIG. 1(B), the spiral portion 43 included in the strut 41 is provided with a pair of linear portions 45a and 45b that extends to be inclined at a predetermined angle with respect to the axial direction of the stent body 30, and a curved portion (turned-back portion) 48 provided between the pair of linear portions 45a and 45b. One spiral portion 43 is configured, with the linear portions 45a and 45b and the curved portion 48 formed to be repeated along a predetermined length. A plurality of spiral portions 43 are provided side by side in series in the axial direction of the stent body 30, and thereby the entire stent 10 forms one spiral body. Note that there are no particular limitations on the number of spiral portions 43, the number of curved portions 48, the number of connection sections 60, or the like.

FIGS. 2(A) and 2(B) illustrate cross sections of the strut 41.

The stent 10 includes a biodegradable drug-coating portion 70 formed on the strut 41, and an adhesion improving portion (adhesion improving layer) 80 formed between the strut 41 and the drug-coating portion 70. As illustrated in FIG. 2(A), for example, it is possible for the drug-coating portion 70 not to be formed on an inner surface 41a of the strut 41, but to be formed on an outer surface 41b of the strut 41 and on a part of a side surface 41c of the strut 41. For example, as illustrated in FIG. 2(B), it is possible for the drug-coating portion to be formed only on the outer surface 41 of the strut 41. In a case where the drug-coating portion 70 is formed on the outer surface 41b and the side surface 41c of the strut 41, the adhesion improving portion 80 is formed on the outer surface 41b and the side surface 41c of the strut 41, similar to the drug-coating portion 70. In a case where the drug-coating portion 70 is formed only on the outer surface 41b of the strut 41, the adhesion improving portion is formed only on the outer surface 41b of the strut 41, similar to the drug-coating portion 70. The drug-coating portion 70 is not formed on the inner surface 41a (inner surface side of the stent 10) of the strut 41, and thereby it is possible to prevent formation of a neointima from being inhibited on the inner surface 41a. Therefore, it is possible to prevent the stenosis or obstruction from occurring due to a thrombus produced inside the stent 10.

Next, constituent materials of the portions of the stent 10 according to the embodiment will be described.

Both of the stent body 30 and the drug-coating portion 70 contain biodegradable (co)polymers. Here, there is no particular limitation on the biodegradable (co)polymers that can be used for the stent body 30 and the drug-coating portion 70, and known biodegradable (co)polymers such as those disclosed in JP-T-2011-528275, JP-T-2008-514719, International Publication No. 2008-1952, and JP-T-2004-509205 can be used. Specifically, examples thereof include (1) a polymer selected from the group consisting of aliphatic polyester, polyester, polyanhydride, polyorthoester, polycarbonate, polyphosphazene, polyphosphate ester, polyvinyl alcohol, polypeptide, polysaccharide, proteins, and cellulose, (2) a copolymer consisting of one or more monomers of which (1) above consists. In other words, it is preferable that the stent body 30 and the drug-coating portion 70 are independent from each other, and contain a polymer selected from the group consisting of aliphatic polyester, polyester, polyanhydride, polyorthoester, polycarbonate, polyphosphazene, polyphosphate ester, polyvinyl alcohol, polypeptide, polysaccharide, proteins, and cellulose, and at least one of biodegradable (co)polymers selected from the group consisting of copolymers that consist of one or more monomers of which the polymers consist. Note that, hereinafter, the polymers and the copolymers are collectively referred to as a “biodegradable (co)polymer”.

Here, there is no particular limitation on the aliphatic polyester, and examples thereof include polylactic acid (PLA) such as poly-L-lactic acid, poly-D-lactic acid, or poly-DL-lactic acid, polyglycolic acid (PGA), polyhydroxybutyric acid, polyhydroxyvaleric acid, polyhydroxypentanoic acid, polyhydroxyhexanoic acid, polyhydroxyheptanoic acid, polycaprolactone, polycarbonate trimethylene, polydioxanone, polymalic acid, polyethylene adipate, polyethylene succinate, polybutylene adipate, or polybutylene succinate. In addition, there is no particular limitation on the polycarbonate, and an example thereof includes a tyrosine-polycarbonate or the like.

Alternatively, the stent body 30 and the drug-coating portion 70 may contain a copolymer obtained by arbitrary copolymerization of monomers of which the polymers consist. Here, there is no particular limitation on the copolymer. Specifically, examples of the copolymer include a lactic acid-caprolactone copolymer, a caprolactone-glycolic acid copolymer, poly(lactide-co-glycolide) (PLGA), polyanhydride, polyorthoester, poly(N-(2-hydroxypropyl)methacrylamide), DLPLA-poly(dl-lactide), LPLA-poly(l-lactide), PGA-polyglycolide, PDO-poly(dioxanone), PGA-TMC-poly (glycolide-co-trimethylene carbonate), PGA-LPLA-poly(l-lactide-co-glycolide), PGA-DLPLA-poly(dl-lactide-co-glycolide), LPLA-DLPLA-poly(l-lactide-co-dl-lactide), and PDO-PGA-TMC-poly (glycolide-co-trimethylene carbonate-co-dioxanone), polyanhydride esters (PAE)-salicylate (for example, a polymer obtained when salicylate is coupled to both ends of polylactide anhydride or polyadipic acid) obtained when salicylate is chemically introduced into a polymer main chain, or the like.

The polymers and the copolymers may be individually used, may be used in combinations of two or more polymers or copolymers, or may be used in combinations of one or more polymers and one or more copolymers, respectively. In addition, the polymers and the copolymers may be manufactured by combinations, respectively, or commercialized products thereof may be used. There is no limitation on a composition method; however, it is possible to apply a known method as is or to apply an appropriately modified method. For example, the polylactic acid (PLA), the polyglycolic acid (PGA), or poly(lactic-co-glycolic acid) (PLGA) is obtained through dehydration polycondensation of a material having a required structure which is selected from L-lactic acid, D-lactic acid, and glycolic acid as a raw material. Preferably, it is possible to obtain the polymer or the copolymer through ring-opening polymerization of a material having the required structure which is selected from lactide as a cyclic dimer of lactic acid or glycolide as a cyclic dimer of glycolic acid. Examples of the lactide include L-lactide as a cyclic dimer of L-lactic acid, D-lactide as a cyclic dimer of D-lactic acid, or DL-lactide as a racemic mixture of D-lactide and L-lactide and meso-lactide obtained by cyclic dimerization of D-lactic acid and L-lactic acid. However, it is possible to use any lactide.

There is no particular limitation on weight-average molecular weight of the biodegradable (co)polymer, as long as it is possible to exhibit an appropriate biodegradation rate. Specifically, it is preferable that the weight-average molecular weight of the biodegradable (co)polymer is preferably 10,000 or larger. In other words, it is preferable that the stent body 30 and the drug-coating portion 70 contain biodegradable (co)polymers having the weight-average molecular weight of 10,000 or larger. The weight-average molecular weight of the (co)polymer is preferably 10,000 to 1,000,000, and more preferably 20,000 to 500,000. Note that examples of a measurement method of the weight-average molecular weight include a gel permeation chromatography (GPC), a light scattering method, a viscometric method, or mass spectrometry (TOFMASS or the like).

Among the biodegradable (co)polymers, preferably, polylactic acid (PLA), polyglycolic acid (PGA), polycaprolactone, a lactic acid-caprolactone copolymer, a caprolactone-glycolic acid copolymer, lactic acid-glycolic acid copolymer (PLGA), tyrosine-polycarbonate, or polyanhydride esters (PAE)-salicylate is used. This is because the (co)polymers have high biocompatibility and it is easy to control the degradation thereof in the living body.

The drug-coating portion 70 is more rapidly biodegraded than the stent body 30 (that is, a biodegradation rate of the stent body 30<a biodegradation rate of the drug-coating portion 70). Here, the stent body 30 may be biodegraded in a period of about 6 months to 9 months, about 6 months to 12 months, about 9 months to 15 months, about 9 months to 12 months, about one year to two years, or about three years to four years. In addition, the drug-coating portion 70 may be biodegraded in a period of about 45 days to 120 days, about 45 days to 90 days, about 60 days to 120 days, about 60 days to 90 days, or about 12 months to 18 months.

Here, there is no particular limitation on a magnitude relationship between biodegradation rates of the stent body 30 and the drug-coating portion 70 as long as such a relationship described above is satisfied. Preferably, the stent body 30 maintains a constant expansion retention force for a certain period (for example, 6 months or longer) (maintains a shape at the beginning of the expansion), whereas the drug-coating portion 70 is relatively early biodegraded in a certain period (for example, about three months) after the stent 10 indwells in a predetermined site. Specifically, it is preferable that (a) the drug-coating portion 70 contains a biodegradable (co)polymer having a biodegradation rate at which 10% by mass or less of an amount of the (co)polymer remains in the drug-coating portion 70 when the expansion retention force of the stent 10 is 0.2 N/mm after the stent is immersed in a phosphate buffered physiological salt solution having a temperature of 37° C., with respect to an amount of the (co)polymer of the drug-coating portion 70 before the immersion; and/or (b) the drug-coating portion 70 contains a biodegradable (co)polymer having a biodegradation rate at which 5% by mass or less of an amount of the (co)polymer remains in the drug-coating portion 70 within 6 months after the stent 10 is immersed in the phosphate buffered physiological salt solution having the temperature of 37° C., with respect to the amount of the (co)polymer of the drug-coating portion 70 before the immersion.

There is no particular limitation on an adjustment method of the biodegradation rate, in order for the biodegradable (co)polymer used for the stent body 30 and the drug-coating portion 70 to be degraded at a biodegradation rate that satisfies the relationship described above, for example, examples of the method include (i) a method for adjusting a molecular weight of the biodegradable (co)polymer, (ii) a method for controlling a composition of the biodegradable (co)polymer, (iii) a method for controlling a glass-transition temperature (Tg) of the biodegradable (co)polymer, (iv) a method for controlling crystallinity of the biodegradable (co)polymer, or the like. Among the method described above, (i) and (ii) are preferable. In the method of (i), in normal, when the molecular weight (weight-average molecular weight) of the biodegradable (co)polymer increases, the biodegradation rate is slow. Therefore, it is preferable that the molecular weight (weight-average molecular weight) of the biodegradable (co)polymer, which is used in the stent body 30, is adjusted to be larger than the molecular weight (weight-average molecular weight) of the biodegradable (co)polymer, which is used in the drug-coating portion 70. Here, there is no particular limitation on a magnitude relationship between the molecular weights (weight-average molecular weights) of the biodegradable (co)polymers which are used in the stent body 30 and the drug-coating portion 70, and the relationship is appropriately controlled, depending on a difference from a desired biodegradation rate. In addition, a content of the biodegradable (co)polymer having low molecular weight (for example, 10,000 or smaller), which is contained in the drug-coating portion 70, may be adjusted to be larger than an amount of the copolymer contained in the stent body 30 (for example, 1% by mass or larger, and preferably at a percentage of 1% to 50% by mass). In other words, according to a preferred embodiment, a content of the biodegradable (co)polymer having the weight-average molecular weight of 10,000 or smaller which is contained in the drug-coating portion 70 is 1% by mass or larger of a content of the biodegradable (co)polymer having the weight-average molecular weight of 10,000 or smaller which is contained in the stent body 30. Here, there is no particular limitation on the upper limit of the difference in the contents, and the upper limit is appropriately controlled, depending on the difference from the desired biodegradation rate.

In addition, in the method of (ii) described above, for example, a biodegradable (co)polymer having a relatively slow biodegradation rate is used in the stent body 30, and a biodegradable (co)polymer having a relatively fast biodegradation rate is used in the drug-coating portion 70 in some cases. Here, when glycolic acid or a caprolactone-derived constituting unit is introduced, the biodegradation rate increases. Therefore, it is preferable that polylactic acid or a biodegradable (co)polymer, which contains a large composition of lactic acid-derived constituting unit, such as a glycolic acid-lactic acid copolymer that contains 90 mol % or more of lactic acid with respect to all monomers, a caprolactone-glycolic acid copolymer that contains 96 mol % or more of lactic acid with respect to all monomers, or a caprolactone-lactic acid copolymer that contains 96 mol % or more of lactic acid with respect to all monomers, is used in the stent body 30, whereas a biodegradable (co)polymer having relatively fast biodegradation rate, such as polyglycolic acid, polycaprolactone, a glycolic acid-lactic acid copolymer that contains 10 mol % or more of polyglycolic acid with respect to all monomers, a caprolactone-glycolic acid copolymer that contains 4 mol % or more of polycaprolactone with respect to all monomers, or a caprolactone-lactic acid copolymer that contains 4 mol % or more of polycaprolactone with respect to all monomers, is used in the drug-coating portion 70. Of the examples, it is preferable that the stent body 30 and the drug-coating portion 70 contain biodegradable (co)polymers that consist of the same constituting unit, but have different compositions from each other. The biodegradable (co)polymers consisting of the same constituting unit are used in the stent body 30 and the drug-coating portion 70, and thereby it is possible to improve adhesion between the stent body 30 and the drug-coating portion 70 (possible to more effectively reduce or prevent separation of the drug-coating portion 70).

In addition, the drug-coating portion 70 may contain the biodegradable (co)polymer exposed to irradiation in advance (for example, gamma irradiation or electron-beam irradiation). In general, when the (co)polymer is exposed to irradiation, coupling in the (co)polymer is likely to be cut, and the biodegradation rate increases. Therefore, even in a case where the biodegradable (co)polymers having the same composition are used, it is possible to adjust the biodegradation rate such that the biodegradation rate satisfies the preferred relationship, by exposing, to irradiation in advance, the biodegradable (co)polymer that is used in the drug-coating portion 70. In addition, the biodegradable (co)polymers having the same composition are used in the stent body 30 and the drug-coating portion 70, and thereby it is possible to improve the adhesion.

There is no particular limitation on thicknesses of the stent body 30 and the drug-coating portion 70, and the thicknesses are set within a range in which performance of the stent body 30, such as reachability (delivery property) to a lesion or stimulation to a vascular wall, is not remarkably reduced, or within a range in which effects of release of the drug are sufficiently exhibited in the drug-coating portion 70. Specifically, an average thickness of the stent body 30 and the drug-coating portion 70 is preferably 1 to 75 μm, more preferably, 2 to 30 μm, and still more preferably, 3 to 10 μm. With the thickness described above, when the stent 10 indwells in a body lumen, the drug is gradually released in a highly effective manner, and it is possible to reduce or prevent an occurrence of restenosis because there is less increase in an outer diameter of the stent 10, it is possible to reduce a concern about interference occurring when the stent 10 reaches the lesion, and a vascular wall is not stimulated.

The drug-coating portion 70 contains drug, in addition to the biodegradable (co)polymer. Here, there is no particular limitation on the drug, as long as the drug is for reducing an occurrence of the stenosis or obstruction in a vessel system which is caused during a procedure of indwelling of the stent 10 in the lesion, and it is possible to arbitrarily select one. Specifically, examples of the drug include an anticancer drug, an immunosuppressive drug, an antibiotic drug, an antithrombogenic drug, an HMG-CoA reductase inhibitor, an ACE inhibitor, a calcium antagonist, an antihyperlipidemic drug, an integrin inhibitor, an anti-allergic drug, antioxidant, an GPIIbIIIa antagonist, a retinoid, lipid-improving drug, an antiplatelet drug, an anti-inflammatory drug, and the like. It is preferable that such drugs suppress behavior of cells in tissue of the lesion and can perform medical treatment on the lesion.

There is no particular limitation on the anticancer drug, and examples thereof include, preferably, paclitaxel, docetaxel, vinblastine, vindesine, irinotecan, pirarubicin, or the like.

There is no particular limitation on the immunosuppressive drug, and examples thereof include, preferably, sirolimus, a sirolimus derivative such as everolimus, pimecrolimus, zotarolimus or biolimus (for example, biolimus A9 (registered trademark)), tacrolimus, azathioprine, cyclosporine, cyclophosphamide, mycophenolate mofetil, gusperimus, or the like.

There is no particular limitation on the antibiotic drug, and examples thereof include, preferably, mitomycin, adriamycin, doxorubicin, actinomycin, daunorubicin, idarubicin, pirarubicin, aclarubicin, epirubicin, ginostatin stimulamer, or the like.

There is no particular limitation on the antithrombogenic drug, and examples thereof include, preferably, aspirin, ticlopidine, argatroban, or the like.

There is no particular limitation on the HMG-CoA reductase inhibitor, and examples thereof include, preferably, cerivastatin, cerivastatin sodium, atorvastatin, pitavastatin, fluvastatin, fluvastatin sodium, simvastatin, lovastatin, or the like.

There is no particular limitation on the ACE inhibitor, and examples thereof include, preferably, quinapril, trandolapril, temocapryl, delapril, enalapril maleate, captopril, or the like.

There is no particular limitation on the calcium antagonist, and examples thereof include, preferably, nifedipine, nilvadipine, benidipine, nisoldipine, or the like.

There is no particular limitation on the antihyperlipidemic drug, and an example thereof includes, preferably, probucol.

There is no particular limitation on the integrin inhibitor, and an example thereof includes, preferably, AJM300.

There is no particular limitation on the anti-allergic drug, and an example thereof includes, preferably, tranilast.

There is no particular limitation on the antioxidant, and examples thereof include, preferably, α-tocopherol, catechin, dibutylhydroxytoluene, or butylhydroxyanisole.

There is no particular limitation on the GPIIbIIIa antagonist, and an example thereof includes, preferably, abciximab.

There is no particular limitation on the retinoid, and an example thereof includes, preferably, all-trans-retinoic acid.

There is no particular limitation on the lipid-improving drug, and an example thereof includes, preferably, eicosapentaenoic acid.

There is no particular limitation on the antiplatelet drug, and examples thereof include, preferably, ticlopidine, cilostazol, or clopidogrel.

There is no particular limitation on the anti-inflammatory drug, and an example thereof includes, preferably, a steroid such as dexamethasone or prednisolone.

The drug-coating portion 70 may contain only one of the drugs described above, or may contain two or more different drugs. In a case where two or more drugs are contained, a combination of the drugs may be appropriately selected from the drugs described above as necessary. According to the present disclosure, the drugs are preferably the immunosuppressive drug or anticancer drug, or more preferably the immunosuppressive drug. In other words, it is preferable that the drug-coating portion 70 contains the immunosuppressive drug. It is more preferable that the immunosuppressive drug such as sirolimus, a sirolimus derivative such as everolimus, pimecrolimus, zotarolimus or biolimus (for example, biolimus A9 (registered trademark)), tacrolimus, azathioprine, cyclosporine, cyclophosphamide, mycophenolate mofetil, gusperimus, or the like is contained.

Here, there is no particular limitation on a content of the drug in the drug-coating portion 70, as long as desired drug efficacy is obtained with the amount. Specifically, a composition ratio (mass ratio) of the biodegradable (co)polymer and the drug in the drug-coating portion 70 is preferably 1:99 to 99:1, and more preferably 5:95 to 80:20. According to the composition, it is possible to effectively release an appropriate amount of the drug for a predetermined period.

There is no particular limitation on a method for forming the drug-coating portion 70, and it is possible to apply the conventional coating method or an appropriately modified method. Specifically, a mixture is prepared by mixing the biodegradable (co)polymer, the drug, and an appropriate solvent as necessary, and it is possible to apply a method in which the mixture is applied.

The adhesion improving portion 80, which is provided between the stent body 30 and the drug-coating portion 70 so as to improve the adhesion between the stent body 30 and the drug-coating portion 70, contains the biodegradable (co)polymers; however, specific examples of the biodegradable (co)polymers are the same as described above, the description thereof is omitted. It is preferable that the adhesion improving portion 80 contains the same biodegradable (co)polymer used in the drug-coating portion 70. In this manner, it is possible to more improve the adhesion between the adhesion improving portion 80 and the drug-coating portion 70 (possible to effectively reduce or prevent separation of the drug-coating portion 70). In addition, when the drug-coating portion 70 is completely degraded, the adhesion improving portion 80 is also degraded substantially in the same period. Therefore, after the degradation of the drug-coating portion 70, the stent body 30 is not inhibited from being degraded. The adhesion improving portion 80 does not practically contain the drug (a content of the drug is 5% by mass or less with respect to the adhesion improving portion 80 in terms of a solid content).

There is no particular limitation on a thickness of the adhesion improving portion 80 in a case where the adhesion improving portion 80 is provided between the stent body 30 and the drug-coating portion 70, and the thickness is set within a range in which performance of the stent body 30, such as reachability (delivery property) to the lesion area or stimulation to a vascular wall, is not remarkably reduced. Specifically, an average thickness of the adhesion improving portion 80 is preferably 1 to 50 μm, more preferably, 2 to 20 μm, and still more preferably, 2.5 to 10 μm. With the thickness described above, it is possible to improve the adhesion between the stent body 30 and the drug-coating portion 70.

In addition, there is no particular limitation on dimensions of portions of the stent body 30, an outer diameter is preferably 2.1 to 30 mm, more preferably 3.0 to 20 mm, and a length in the axial direction is preferably 5 to 250 mm, and more preferably 8 to 200 mm.

Next, an example of a preferred period until the each portion of the stent 10 according to the embodiment are degraded, and an example of a temporal change in the expansion retention force are described with reference to FIG. 3.

As illustrated in FIG. 3, the stent 10 according to the present embodiment has a configuration in which, in an expansion retention period (T4) during which an expansion retention force of the deformed diameter-expanded stent body 30 that acts on an inner wall of a lumen thereof is maintained, and before 60% of a degradation period (T5) from indwelling of the stent body 30 in the living body to complete degradation thereof elapses, the drug-coating portion 70 is degraded. For example, it is possible to set the degradation period (T5) to be 24 months (about two years).

In the embodiment, it is possible to define the expansion retention period of the stent 10 to be a period in which the radial force of the stent 10 is maintained to be 0.2 N/mm or larger. For example, it is possible to confirm the expansion retention period of the stent 10 in a state of indwelling in a blood vessel of the living body, as follows. It is possible to set the expansion retention period as a period (time) from a time point when immersion of the stent starts after the stent 10 is immersed in the phosphate buffered physiological salt solution having a temperature of 37° C. and the radial force is measured after a predetermined period elapses, to a time point when the radial force is smaller than 0.2 N/mm.

In addition, it is possible to obtain the radial force by evenly compressing the outer peripheral side of the stent 10 and measuring the maximum value of a force generated when the expanded diameter is compressed by 50%, by performing comparison to the stent 10 which is in a state in which no external force is applied.

In addition, it is possible to employ a configuration in which the drug-coating portion 70 is preferably degraded until 25% of the degradation period (T5) elapses. In a case where the degradation period (T5) is 24 months, it is possible to employ a configuration in which the drug-coating portion 70 is degraded within 6 months (about a half year).

In addition, more preferably, it is possible to employ a configuration in which the drug-coating portion 70 is degraded before an initial expansion retention force of the stent body 30 (stent 10) which acts on the inner wall of the lumen at indwelling start time (T1) starts to decrease. In the case where the degradation period (T5) is 24 months, it is possible to set the time “when the initial expansion retention force starts to decrease” as the time (T3) when seven to eight months elapse after the indwelling start time (T1). Note that it is possible to set the initial expansion retention force to be 0.5 to 4.0 N/mm although depending on a state of the lumen or a disorder as the treatment target.

In addition, it is possible to employ a configuration in which the stent body 30 loses the expansion retention force until 50% of the degradation period (T5) elapses. In the case where the degradation period (T5) is 24 months, it is possible to set the time “when the expansion retention force is lost” as the time (T4) when 12 months elapse after the indwelling start time (T1).

In addition, it is possible to employ a configuration in which the stent body 30 maintains the expansion retention force to 50% or larger of the initial expansion retention force that acts on the inner wall of the lumen at the indwelling start time until 25% of the degradation period (T5) elapses. In the case where the degradation period (T5) is 24 months, it is possible to employ a configuration in which the stent body 30 maintains the expansion retention force of 50% or larger than the initial expansion retention force until 6 months elapse from the indwelling start time (T1).

Next, a change in a state of the stent 10 when the stent 10 having such a configuration as described above indwells in the lumen (blood vessel 100) in the living body will be described with reference to FIGS. 4 to 9.

First, as illustrated in FIG. 4, the stent 10 is delivered into a lumen 110 of the blood vessel 100 in which a stenosed site 120 is formed, in a state in which a balloon (not illustrated) provided in a balloon catheter is crimped. Note that it is possible to use a known balloon catheter such as a rapid exchange type, an over-the-wire type, or the like as a balloon catheter for delivering the stent 10 into the living body, for example.

Next, as illustrated in FIGS. 5(A) and 5(B), the stent 10 causes the mounted balloon to dilate, and thereby the stent 10 is deformed to have an expanded diameter. The deformed diameter-expanded stent 10 indwells in a state in which the expansion retention force acts on the inner wall 101 of the blood vessel 100. The balloon is appropriately deflated and is removed from the blood vessel 100.

As illustrated in FIGS. 6(A) and 6(B), when a predetermined period (T2 in FIG. 3) elapses after the indwelling start time of the stent 10, the drug-coating portion 70 and the adhesion improving portion 80 are degraded. Note that elution of the drug from the drug-coating portion 70 is ended before the drug-coating portion 70 is degraded.

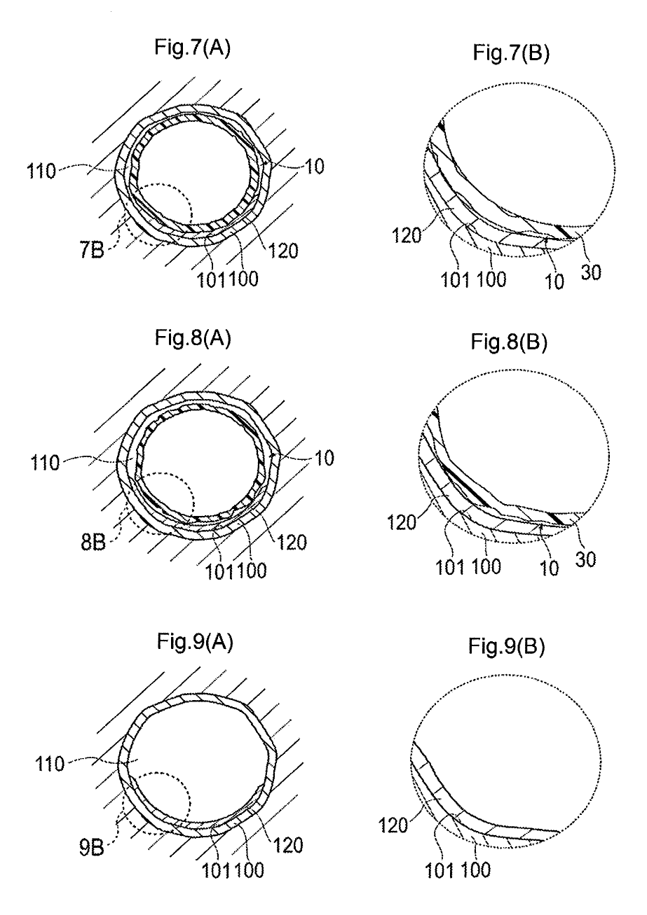

As illustrated in FIGS. 7(A) and 7(B), when a predetermined period elapses after the drug-coating portion 70 is degraded, a decrease (reduction) in the expansion retention force of the stent body 30 starts (at the time T3 in FIG. 3) depending on the progress of the degradation of the stent body 30.

As illustrated in FIGS. 8(A) and 8(B), when a predetermined period elapses and the degradation of the stent body 30 proceeds, the expansion retention force of the stent body 30 is lost (at the time T4 in FIG. 3). Note that the “loss of the expansion retention force” means a state in which the stent body 30 does not have an expansion force acting on to widen the lumen 110 of the blood vessel 100 regardless of direct contact or non-contact of the stent body 30 with the inner wall 101 of the blood vessel 100.

Then, when a predetermined period further elapses, as illustrated in FIGS. 9(A) and 9(B), the degradation of the stent body 30 (stent 10) is ended (at the time T5 in FIG. 3).

As described above, in the stent 10 according to the present disclosure, the desired drug efficacy by the drug-coating portion 70 is exhibited and the drug-coating portion 70 is degraded in the expansion retention period during which the stent body 30 maintains the expansion retention force (radial force). In addition, after the desired drug efficacy is exhibited, the drug-coating portion 70 is rapidly degraded before the stent body 30 is degraded, whereas the stent body 30 maintains the expansion retention force over the predetermined period even after the drug-coating portion 70 is degraded. Hence, it is possible to provide the biodegradable stent that can suitably maintain, over the desired period, a state in which the lumen (blood vessel 100) is widened, and improves treatment effect due to suitable exhibition of drug efficacy.

Hereinafter, more specific examples are described; however, the present invention is not limited thereto.

### Example 1

A stent of Example 1 is configured to include a stent body (material: PLLA) that has a cylindrical shape with an outer diameter of 2.0 mm and a length of 18 mm in the axial direction and is formed by a linear configurational element (with a width of 0.1 mm) having a substantially rhombic notch (a portion represented by a dashed line portion 49 in FIG. 1(B)), and a drug-coating portion containing a mixture at a ratio of 1 to 1 by a weight ratio of the lactic acid-caprolactone copolymer and sirolimus.

The drug-coating portion of the stent of Example 1 is completely degraded within four months by hydrolysis. The stent body of the stent of Example 1 maintains 70% or larger of weight measured before the indwelling, after five months elapse after the start of the indwelling, and the radial force is 1.5 N/mm. In addition, the degradation period of the stent of Example 1 is four years.

### Example 2

A stent of Example 2 is configured to include a stent body (material: PLLA) that has a cylindrical shape with an outer diameter of 2.0 mm and a length of 18 mm in the axial direction and is formed by a linear configurational element (with a width of 0.1 mm) having a substantially rhombic notch (a portion represented by a dashed line portion 49 in FIG. 1(B)), and a drug-coating portion containing a mixture at a ratio of 1 to 1 by a weight ratio of the PLGA and sirolimus.

The drug-coating portion of the stent of Example 2 is completely degraded within five months by hydrolysis. The stent body of the stent of Example 2 maintains 70% or larger of weight measured before the indwelling, after five months elapse after the start of the indwelling, and the radial force is 1.5 N/mm. In addition, the degradation period of the stent of Example 2 is four years.

The detailed description above describes features and aspects of embodiments of a biodegradable stent disclosed by way of example. The invention is not limited, however, to the precise embodiments and variations described. Changes, modifications and equivalents can be employed by one skilled in the art without departing from the spirit and scope of the invention as defined in the appended claims. It is expressly intended that all such changes, modifications and equivalents which fall within the scope of the claims are embraced by the claims.

What is claimed is:

1. A biodegradable stent that is degraded in a living body, the biodegradable stent comprising:
a stent body that is made of a biodegradable material and is deformed to have an expanded diameter in the living body; and
a biodegradable drug-coating portion formed on the stent body,
wherein the drug-coating portion is degraded, in an expansion retention period during which an expansion retention force of the deformed diameter-expanded stent body that acts on an inner wall of a lumen thereof is maintained, and before 60% of a degradation period from indwelling of the stent body in the living body to complete degradation thereof elapses.

2. The biodegradable stent according to claim 1,
wherein the drug-coating portion is degraded until 25% of the degradation period elapses.

3. The biodegradable stent according to claim 1,
wherein the drug-coating portion is degraded before an initial expansion retention force of the stent body which acts on the inner wall of the lumen at the beginning of indwelling starts to decrease.

4. The biodegradable stent according to claim 1,
wherein the stent body loses the expansion retention force until 50% of the degradation period elapses.

5. The biodegradable stent according to claim 1,
wherein the stent body maintains the expansion retention force to 50% or higher of the initial expansion retention force that acts on the inner wall of the lumen at indwelling start time until 25% of the degradation period elapses.

6. The biodegradable stent according to claim 1,
wherein the degradation period is 24 months.

7. The biodegradable stent according to claim 1,
wherein the stent body and the drug-coating portion are independent from each other, and contain a polymer selected from the group consisting of aliphatic polyester, polyester, polyanhydride, polyorthoester, polycarbonate, polyphosphazene, polyphosphate ester, polyvinyl alcohol, polypeptide, polysaccharide, proteins, and cellulose, and at least one of biodegradable (co)polymers selected from the group consisting of copolymers that consist of one or more monomers of which the polymers consist.

8. The biodegradable stent according to claim 7,
wherein the drug-coating portion contains a biodegradable (co)polymer having a biodegradation rate at which 10% by mass or less of an amount of the (co)polymer remains in the drug-coating portion when the expansion retention force is 0.2 N/mm after the stent is immersed in a phosphate buffered physiological salt solution having a temperature of 37° C., with respect to an amount of the (co)polymer in the drug-coating portion before the immersion.

9. The biodegradable stent according to claim 7,
wherein the drug-coating portion contains a biodegradable (co)polymer having a biodegradation rate at which 5% by mass or less of an amount of the (co)polymer remains in the drug-coating portion within 6 months after the stent is immersed in a phosphate buffered physiological salt solution having a temperature of 37° C., with respect to the amount of the (co)polymer in the drug-coating portion before the immersion.

10. The biodegradable stent according to claim 7,
wherein the stent body and the drug-coating portion contain biodegradable (co)polymers having weight-average molecular weight of 10,000 or larger.

11. The biodegradable stent according to claim 7,
wherein the stent body and the drug-coating portion contain biodegradable (co)polymers that have the same constituting unit, but have different compositions from each other.

12. The biodegradable stent according to claim 7,
wherein the drug-coating portion contains a biodegradable (co)polymer that is exposed to irradiation in advance.

13. The biodegradable stent according to claim 7,
wherein a content of a biodegradable (co)polymer having the weight-average molecular weight of 10,000 or smaller which is contained in the drug-coating portion is 1% by mass or higher of a content of a biodegradable (co)polymer having the weight-average molecular weight of 10,000 or smaller which is contained in the stent body.

14. The biodegradable stent according to claim 1,
wherein the drug-coating portion contains an immunosuppressive drug.

15. The biodegradable stent according to claim 14,
wherein the immunosuppressive drug is at least one selected from the group consisting of sirolimus, sirolimus derivative such as everolimus, pimecrolimus and zotarolimus, biolimus, tacrolimus, azathioprine, cyclosporine, cyclophosphamide, mycophenolate mofetil, and gusperimus.

16. The biodegradable stent according to claim 1,
wherein the drug-coating portion is formed only on an outer surface of a strut formed on the stent body, or formed on the outer surface of the strut and only on at least a part of a side surface of the strut.

17. The biodegradable stent according to claim 1, further comprising:
an adhesion improving portion that is made of a biodegradable material different from a material, of which the stent body is made, and improves adhesion of the drug-coating portion to the stent body between the stent body and the drug-coating portion,
wherein the adhesion improving portion is degraded before the stent body is degraded.

18. A biodegradable stent that is degraded in a living body, the biodegradable stent comprising:
a stent body that is made of a biodegradable material and is deformed to have an expanded diameter in the living body; and
a biodegradable drug-coating portion formed on the stent body,
wherein the stent body and the drug-coating portion are independent from each other, and contain a polymer selected from the group consisting of aliphatic polyester, polyester, polyanhydride, polyorthoester, polycarbonate, polyphosphazene, polyphosphate ester, polyvinyl alcohol, polypeptide, polysaccharide, proteins, and cellulose, and at least one of biodegradable (co)polymers selected from the group consisting of copolymers that consist of one or more monomers of which the polymers consist, and
wherein the drug-coating portion contains a biodegradable (co)polymer having a biodegradation rate at which 10% by mass or less of an amount of the (co)polymer remains in the drug-coating portion when the expansion retention force is 0.2 N/mm after the stent is immersed in a phosphate buffered physiological salt solution having a temperature of 37° C., with respect to an amount of the (co)polymer in the drug-coating portion before the immersion.

19. The biodegradable stent according to claim 18,
wherein the drug-coating portion contains a biodegradable (co)polymer having a biodegradation rate at which 5% by mass or less of an amount of the (co)polymer remains in the drug-coating portion within 6 months after the stent is immersed in a phosphate buffered physiological salt solution having a temperature of 37° C., with respect to the amount of the (co)polymer in the drug-coating portion before the immersion.
